(19) United States
(12) Patent Application Publication
(10) Pub. No.: US 2026/0167952 A1
(43) Pub. Date: Jun. 18, 2026

(54) METHODS AND COMPOSITIONS TO PROTECT DNA DURING CELL LYSIS

(71) Applicant: Board of Regents of the University of Nebraska, Lincoln, NE (US)

(72) Inventors: Kristy Kounovsky-Shafer, Kearney, NE (US); Charlie Polen, Lakewood, CO (US); Esmeralda Mendez-Ortiz, Kearney, NE (US)

(21) Appl. No.: 19/355,858

(22) Filed: Oct. 10, 2025

Related U.S. Application Data
(60) Provisional application No. 63/706,012, filed on Oct. 10, 2024.

(51) Int. Cl. C12N 15/10; C12Q 1/6806

(52) U.S. Cl. CPC C12N 15/1003; C12Q 1/6806

(57) ABSTRACT

An encapsulation device is described that can be used to protect and store large DNA molecules during and after cell lysis. Encapsulation devices as described herein typically include an agarose shell; a central well defined within the agarose shell, the central well configured to receive a liquid sample comprising cells and/or the DNA molecules; and a sealing layer of agarose disposed over the central well to encapsulate the liquid sample within the central well. The encapsulation devices are configured such that, upon lysis of the cells within the liquid sample, the DNA molecules remain intact and in solution within the central well for subsequent recovery without melting the agarose shell.

### CROSS-REFERENCE TO RELATED APPLICATIONS

This application claims the benefit of priority under 35 U.S.C. § 119(e) to U.S. Application No. 63/706,012 filed on Oct. 10, 2024. This application is incorporated herein in its entirety.

### FEDERALLY SPONSORED RESEARCH OR DEVELOPMENT

This invention was made with government support under 5P20GM103427 and R15GM155799 awarded by the National Institutes of Health. The government has certain rights in the invention.

### TECHNICAL FIELD

This disclosure generally relates to methods and compositions that protect DNA during cell lysis.

### BACKGROUND

Structural variations alter more base pairs of the human genome than point mutations. These structural variations have been estimated to cause 25-29% of protein-truncating events, which have been linked with many diseases. Many structural variations, such as amplification, deletion, inversions, or translocations, alter genes and non-genetic regions through small repeat extensions. Larger structural variations (>5 kb) are more difficult to discern due to size. Single DNA molecule platforms such as Optical Mapping System, Nanocoding, Oxford Nanopore sequencing, and PacBio rely on complete, long DNA molecules for their system to determine the large structural variations. However, DNA molecules must be extremely long for these systems to have enough information on each side to span the large variations.

### SUMMARY

Determining large structural variations is difficult and time-consuming without long molecules to aid in the genome assembly. One issue is the fragility of large DNA molecules during routine molecular biology techniques. Plug inserts were developed to protect DNA during cell lysis or other molecular biology techniques. However, the plug inserts make accessing the DNA embedded in the agarose plug insert difficult. To combat this, an inverted agarose insert was created and is described herein. A typical plug insert has the cell's solution mixed with agarose, and then the cells are lysed with DNA embedded in the agarose matrix. The inverted agarose insert described herein has the cell solution in the middle and the agarose on the outside.

Multiple concentrations of low melting point agarose were tested to determine the best percentage of agarose to create the inverted insert. The agarose needs to be concentrated enough to allow for easy handling. An exemplary inverted insert was tested with S. cerevisiae cells to show that cells could be lysed inside the inverted insert, allowing the DNA to remain at full length.

In one aspect, encapsulation devices are provided for protecting and storing DNA molecules during cell lysis. Such encapsulation devices typically include an agarose shell; a central well defined within the agarose shell, the central well configured to receive a liquid sample comprising cells and/or the DNA molecules; and a sealing layer of agarose disposed over the central well to encapsulate the liquid sample within the central well, wherein the encapsulation device is configured such that, upon lysis of the cells within the liquid sample, the DNA molecules remain intact and in solution within the central well for subsequent recovery without melting the agarose shell.

In some embodiments, the agarose shell has a concentration between about 0.5% and about 3% (w/v). In some embodiments, the agarose shell has a concentration of about 2% (w/v). In some embodiments, the agarose shell has a thickness of about 0.2 centimeter (cm) to about 0.5 cm. In some embodiments, the agarose shell has a thickness of about 0.30 cm. In some embodiments, the agarose shell has a thickness sufficient to prevent diffusion of the DNA molecules into the agarose shell. In some embodiments, the agarose shell has a rectangular prism shape with a height ranging from about 1 cm to about 2 cm, a width ranging from about 0.5 cm to about 2 cm, and a length ranging from about 0.5 cm to about 2.5 cm. In some embodiments, the agarose shell is in solid form.

In some embodiments, the central well has a volume of about 140 microliters (μL) to about 200 μL. In some embodiments, the central well has a depth of about 0.5 cm to about 2 cm. In some embodiments, the central well is configured to maintain the liquid sample in liquid form.

In some embodiments, the sealing layer of agarose has a thickness of about 0.3 cm to about 1 cm.

In some embodiments, the encapsulation devices further include a holder configured to position the encapsulation device at a defined cutting plane for removal of the sealing layer, thereby enabling access to the central well. In some embodiments, the holder defines a cavity configured to receive the encapsulation device. In some embodiments, the cavity has rectangular dimensions with a length of about 0.5 cm to about 2 cm and a width of about 0.5 cm and about 2 cm. In some embodiments, the cavity has a depth that is less than a height of the agarose shell.

In some embodiments, the DNA molecules that remain intact and in solution have a length of about 100 kilobases (kb) to about 1.5 megabases (Mb). In some embodiments, the DNA molecules that remain intact and in solution have a length of at least about 1 Mb.

In some embodiments, the encapsulation devices further include a plunger configured to push the encapsulation device out of a mold.

In another aspect, methods of isolating DNA molecules are provided. Such methods typically include providing any of the encapsulation devices described herein containing a liquid sample comprising cells and/or the DNA molecules in the central well; contacting the encapsulation device with one or more lysis reagents to lyse the cells within the central well while the agarose shell remains intact; and recovering the DNA molecules in liquid form from the central well without melting the agarose shell.

In some embodiments, recovering the DNA molecules comprises removing a portion of the sealing layer and removing the liquid sample from the central well. In some embodiments, recovering the DNA molecules comprises poking a hole in the insert with a blunt end syringe.

In some embodiments, the lysis reagents comprise Zymolase, beta-mercaptoethanol, EDTA, proteinase K, N-lauroylsarcosine, or any combinations thereof.

In some embodiments, the methods further include rinsing the recovered DNA molecules with a buffer comprising one or both of Tris and EDTA. In some embodiments, the methods further include storing the encapsulation device with the liquid sample in the central well at about 4° C. for at least one week prior to recovery. In some embodiments, the methods further include analyzing the recovered DNA molecules by pulsed-field gel electrophoresis.

In some embodiments, the liquid sample within the central well includes a density-increasing agent to reduce mixing with the sealing layer of agarose during fabrication. In some embodiments, the density-increasing agent is glycerol.

In some embodiments, the DNA molecules have a length of at least 500 kb.

In still another aspect, methods of making an encapsulation device for protecting and storing DNA molecules during cell lysis are provided. Such methods typically include providing a mold configured to form a shell, the mold defining one or more cavities; applying a seal to a bottom opening of the one or more cavities; introducing an agarose solution into the one or more cavities; positioning a mold insert in the one or more cavities containing the agarose solution, the mold insert having a protrusion sized to define a central well; cooling the agarose solution to solidify and form the shell; removing the mold insert from the one or more cavities, thereby forming the central well; dispensing a liquid sample comprising cells and/or DNA molecules into the central well; and applying a sealing layer of agarose to encapsulate the liquid sample, wherein the resulting encapsulation device is configured for subsequent recovery of the DNA molecules from the central well without melting the agarose shell.

In some embodiments, the mold is a rectangular mold. In some embodiments, applying the sealing layer of agarose comprises applying the sealing layer at a temperature of about less than 45° C. In some embodiments, further comprising removing the seal from the bottom opening after cooling the agarose solution. In some embodiments, further comprising removing the solidified shell from the mold after removing the mold insert from the one or more cavities. In some embodiments, removing the shell from the mold comprises ejecting the solidified shell using a mold pusher.

In some embodiments, further comprising, after applying the sealing layer of agarose, placing the encapsulation device in a holder and removing an upper portion of the sealing layer of agarose to expose an opening into the central well. In some embodiments, further comprising, after applying the sealing layer of agarose, storing the encapsulation device in a buffer at about 4° C.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1D shows the mold design. The mold was originally designed in AutoCAD (1A) and then converted into gcode using Cura software (1B). The mold was then printed using an Ultimaker 3 printer (1C) using PLA filament to produce a holder for the inverted insert (1D). For experimentation, the side of the mold was attached to the printer surface and was taped off to avoid any leakage of the agarose solution. Each printed piece used the same steps.

FIG. 2A-2B is a schematic of how the inverted inserts were made. The schematic shows how the inverted inserts were made in the mold from a top-down view (2A) and what it looks like from a side view for the inverted insert (2B). The bottom of the device was taped, and agarose was added to the mold. The mold was placed on ice. Once the gel solidified, the cell solution or DNA solution with loading dye, made with glycerol, was added to the wells, and then warmed agarose (42° C.) was added to the top of the well to complete the inverted insert. The cells were lysed, and the DNA remained behind in the solution.

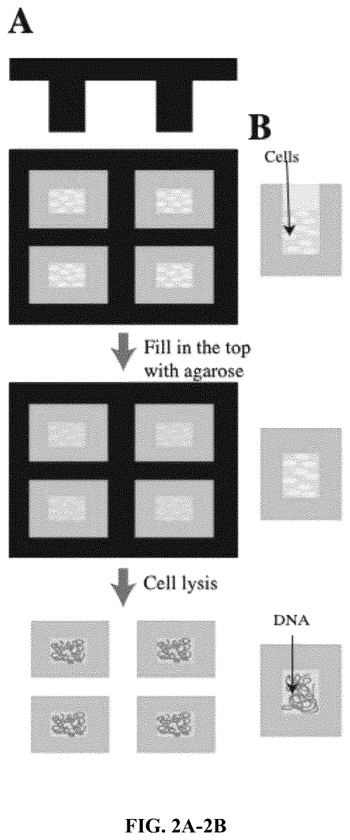

FIG. 3A-3C shows that DNA from the inverted inserts remains full length. The DNA was added to the inverted inserts and stored in 1×TE for one week. The inserts were opened and the solution was removed and added to an agarose gel. The samples were run and imaged on a blue light illuminator and Canon camera. The DNA samples tested were 3A) lambda digested with Acc65I (1.5, 17.1, 29.9 kb fragment lengths), 3B) 1 kb plus ladder (1.5 to 15 kb in size), and 3C) lambda DNA (48.5 kb). The DNA remained full length.

FIG. 4A-4B shows that DNA was retained in the inverted insert for over a year. A 1% agarose gel with DNA was run at 100 V, stained with SYBR gold, and imaged with a blue light transilluminator and Canon camera. (4A) Lambda DNA (48.5 kb) was still full length in the inverted inserts after 1 year. (4B) 1 kb plus ladder from 850 bp to 15.0 kb was still in the inverted insert after 1 year.

FIG. 5 shows that S. cerevisiae DNA stored in the inverted insert was full length. The inverted insert S. cerevisiae solution and S. cerevisiae stored in plug inserts (reference DNA) were run on a pulsed-field 1% agarose gel (Auto algorithm: 200-2200 kb). The gel was stained with SYBR gold for 20 min and then imaged with a blue light transilluminator and Canon EOS T7i camera. The kb markers on the left annotate where strong band patterns should have been in both DNA samples.

FIG. 6A-6D is a schematic of the devices used to make inverted inserts and their dimensions. (6A) The dimensions for the bottom mold for a top-down view and side view. (6B) The top of the mold makes the wells for the inverted insert. The side views are shown. (6C) The side views of the plunger to push the inverted inserts out of the mold are shown. (6D) The side and top view of the holder are shown. This piece helps to hold the insert while cutting off the top of the insert.

FIG. 7A-7B is a schematic for the steps to create an inverted insert. The steps to make the insert in the inverted insert mold (7A) and what happens to the inverted insert (7B). The inverted insert was made by taping the bottom of the insert mold. The liquid agarose was added to each well, and then the top was added to the mold to create wells in the insert. Once the agarose gel was hardened, the top was removed. The cell or DNA solution with loading dye was added to the wells, and agarose was added to the top to seal the insert.

FIG. 8A-8C show the steps for opening the inverted insert. Once the inverted insert was made, it was stored in a buffer until needed. The inverted insert was taken from the 50 mL conical tube (8A) with a glass pipet that was sealed and curved with a Bunsen burner. (8B) The insert was placed into the holder, which was shorter than the height of the insert, and the top was cut off with a glass coverslip. (8C) A pipet was used to remove the DNA solution in the center of the insert for experiments.

FIG. 9 shows the amount of DNA inside the plug insert versus in the solution surrounding the plug insert. Three different low melting point (LMP) agarose concentrations were tested to determine how much DNA remained inside the plug insert. Lambda DNA (2000 ng) was added to the liquid agarose and solidified in an insert mold (not shown). Once the inserts were solid, they were popped out of the insert mold and placed into an Eppendorf tube with buffer for 4 weeks. The amount of DNA in the solution was measured using a NanoDrop spectrophotometer. The insert was digested with beta-agarase to digest the agarose and determine how much DNA remained. The white bars are the DNA percentage in the insert, and the gray bars are the percentage in the solution. Error bars represent the standard deviation. (N=3).

### DETAILED DESCRIPTION

An insert is described that protects large DNA molecules. Previously, agarose plug inserts were designed, in which cells were placed in an agarose block to protect DNA during cell lysis. The cells were then lysed open, and the cellular debris was dialyzed out of the insert. This allowed the long DNA strands to remain intact inside the insert. In addition, a previous method was designed using a 3D-printed device to elute and concentrate lambda DNA from an agarose insert to get the DNA stored in the agarose insert into the solution. This device was later modified to allow larger DNA molecules to be concentrated in the 3-D printed device.

An alternative approach of storing DNA molecules (>1.5 kb) in an inverted insert was developed that does not require heat or chemical methods to access the DNA. Different agarose concentrations were tested to make the inverted inserts, which would allow for reliable storage of large DNA molecules. Lambda DNA, 1 kb plus ladder, and lambda DNA digested with Acc65I were placed into inverted inserts, and the amount of DNA remaining was measured over time. This gave a wide range of DNA sizes tested in the inverted inserts. The DNA retained inside the inverted inserts was compared against how much DNA stayed in the plug inserts over time.

The best concentration for making the inverted inserts was 1.5%, offering the best combination of ease of use and structural integrity. A dynamic range of DNA sizes was stored in the inverted insert, and the DNA remained intact while being stored in the inverted insert for extended periods. S. cerevisiae cells were added to the inverted insert and lysed. The DNA chromosomes remained in the inverted insert and at full length. More DNA remained in the inverted insert compared to the original plug insert. The inverted inserts can store DNA for long periods (>1 yr), keep DNA full length, and allow easy access to the DNA solution.

### Encapsulation Devices

This disclosure describes an encapsulation device for protecting and storing DNA molecules during cell lysis. Encapsulation devices as described herein typically include an agarose shell, a central well defined within the agarose shell configured to receive a liquid sample of cells and/or the DNA molecules, and a sealing layer of agarose disposed over the central well to encapsulate the liquid sample within the central well.

The encapsulation devices described herein are configured such that, upon lysis of the cells within the liquid sample, the DNA molecules remain intact and in solution within the central well for subsequent recovery without needing to melt the agarose shell. The encapsulation devices described herein are designed for recovering intact DNA molecules having a length of about 100 kilobases (kb) up to about 1.5 megabases (Mb) (e.g., DNA molecules having a length of at least about 1 Mb).

Typically, the agarose shell component of an encapsulation device as described herein is in solid form and has an agarose concentration of between about 0.5% and about 3% (w/v) (e.g., about 1% (w/v) and about 2% (w/v); about 2% (w/v) and about 3% (w/v); about 1.5% (w/v) and about 2.5% (w/v); about 2% (w/v)) and a thickness of about 0.2 centimeter (cm) to about 0.5 cm (e.g., about 0.2 cm to about 0.4 cm; about 0.3 cm to about 0.5 cm; about 2.5 cm to about 3.5 cm; about 0.3 cm). As described herein, the agarose shell should have a thickness sufficient to prevent diffusion of the DNA molecules into the agarose shell.

The agarose shell component of an encapsulation device as described herein can have a rectangular prism shape with a height ranging from about 1 cm to about 2 cm (e.g., about 1 cm; about 1.5 cm; about 2 cm), a width ranging from about 0.5 cm to about 2 cm (e.g., about 0.5 cm; about 1 cm; about 1.5 cm; about 2 cm), and a length ranging from about 0.5 cm to about 2.5 cm (e.g., about 0.5 cm; about 1 cm; about 2 cm; about 2.5 cm).

Typically, the central well component of an encapsulation device as described herein is configured to maintain the liquid sample in liquid form. For example, the central well component of an encapsulation device as described herein can hold a volume of about 140 microliters (μL) to about 200 μL (e.g., about 150 μL to about 180 μL; about 160 μL to about 175 μL; about 175 μL to about 200 μL) and can have a depth of about 0.5 cm to about 2 cm (e.g., about 0.5 cm; about 1 cm; about 2 cm).

The sealing layer component of an encapsulation device as described herein is an agarose sealing layer and can have a thickness of about 0.3 cm to about 1 cm (e.g., about 0.5 cm; about 0.6 cm; about 0.8 cm).

An encapsulation device as described herein also can include a holder that is configured to position the encapsulation device at a specific or defined cutting plane. In some instances, the holder is a cavity configured to receive an encapsulation device. Such configuration allows for the removal of the sealing layer to enable access to the central well. For example, a cavity configured to receive an encapsulation device can have rectangular dimensions with a length of about 0.5 cm to about 2 cm (e.g., about 0.75 cm to about 1.5 cm; about 1 cm; about 1.5 cm; about 2 cm) and a width of about 0.5 cm and about 2 cm (e.g., about 0.75 cm to about 1.5 cm; about 1 cm; about 1.5 cm; about 2 cm). It would be appreciated that the cavity can have a depth that is less than a height of the agarose shell.

An encapsulation device as described herein also can include a plunger that is configured to push the encapsulation device out of a mold.

### Methods of Using Encapsulation Devices

The encapsulation devices described herein can be used to isolate DNA molecules having a length of, for example, at least 500 kb (e.g., at least 50,000 kb; at least 0.5 Mb; at least 1 Mb; at least 1.5 Mb).

A liquid sample that includes cells and/or DNA molecules is placed into the central well of an encapsulation device as described herein. It some instances, it may be desirable to include a density-increasing agent (e.g., glycerol) in the liquid sample to reduce mixing with the sealing layer of agarose during fabrication.

Next, the cells contained within the central well can be contacted with one or more lysis reagents to lyse the cells, keeping the agarose shell intact. Lysis reagents are known in the art and include, without limitation, Zymolase, beta-mercaptoethanol, EDTA, proteinase K, N-lauroylsarcosine, or combinations thereof.

The DNA molecules in liquid form then are recovered from the central well without requiring that the agarose shell be melted. For example, a portion of the sealing layer can be removed followed by removal of the liquid sample from the central well or a hole can be made in the insert with, e.g., a blunt end syringe (e.g., 13-18 gauge needle and 1.2-2.4 mm OD) to access the DNA.

The recovered DNA molecules can be rinsed with a buffer that includes, e.g., Tris and/or EDTA. Significantly, the encapsulation device can be stored with the liquid sample in the central well for at least one week at about 4° C. prior to recovering the large DNA molecules. Given the large size of the recovered DNA molecules, further analysis can be performed using pulsed-field gel electrophoresis.

### Methods of Making Encapsulation Devices

The encapsulation devices described herein for protecting and storing DNA molecules during cell lysis can be made as follows.

A mold is provided that forms a shell and defines one or more cavities. In some instances, the mold can be rectangular. A seal is applied to the bottom opening of one or more of the cavities. In some instances, it may be desirable to remove the seal (e.g., from the bottom) after the agarose solution has been cooled. An agarose solution then is introduced into one or more one or more of the cavities and a mold insert having a protrusion sized to define a central well is positioned in one or more of the cavities containing the agarose solution. The agarose solution is cooled to solidify and form the shell and the mold insert is removed from the cavities, thereby forming the central well.

As described herein, the solidified shell can be removed from the mold after the mold insert is removed from the cavities using, for example, a mold pusher. A liquid sample including cells and/or DNA molecules is dispensed into the central well and a sealing layer of agarose is applied to encapsulate the liquid sample. For example, the sealing layer of agarose can be applied at a temperature of about less than about 45° C. (e.g., less than about 42° C.; less than about 37° C.; less than about 30° C.).

After the sealing layer of agarose is applied, the encapsulation device can be placed in a holder and an upper portion of the sealing layer of agarose can be removed to expose an opening into the central well. After the sealing layer of agarose is applied, the encapsulation device can be stored in a buffer at about 4° C. for days, weeks or months. As discussed herein, the encapsulation devices described herein are configured for subsequent recovery of the DNA molecules from the central well without the need to melt the agarose shell.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Supplies

All supplies were purchased from Fischer Scientific (Hampton, NH, USA) unless noted otherwise.

### Example 2—Fabrication of the 3D-Printed Inverted Insert Molds and Accessories

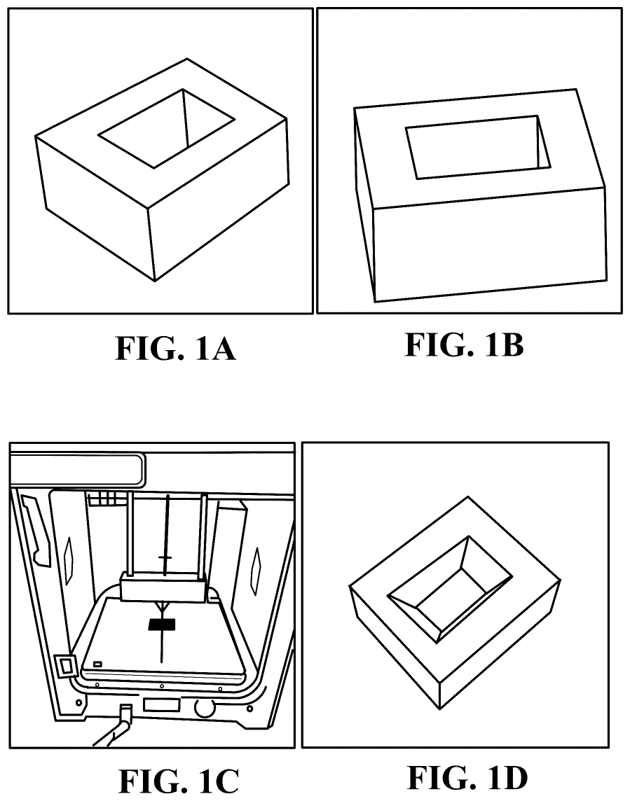

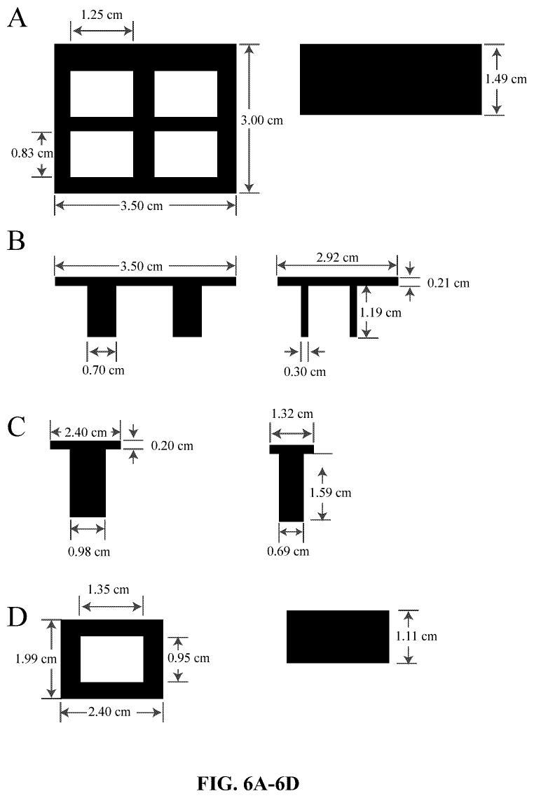

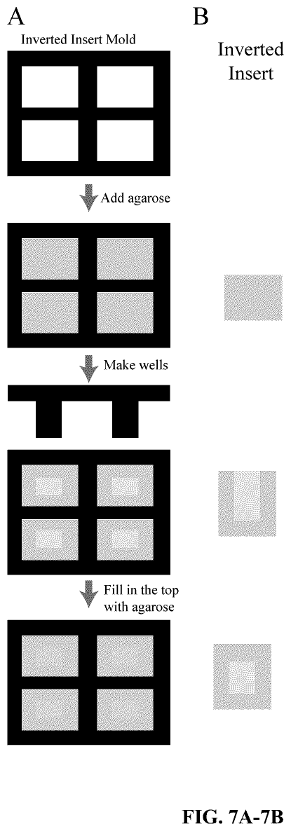

Using a method similar to Masters et al. (2019, Electrophoresis, 40:810-6), the mold for the inverted insert was designed in AutoCAD. The AutoCAD file was then loaded into Cura (Ultimaker software) to determine the adhesion, print speed, and fill-in necessary for printing. The mold was then printed on an Ultimaker 3 printer (FIG. 1; Dynamism, Chicago, IL, USA) using polylactic acid (PLA) filament (Dynamism, Chicago, IL, USA). Each insert mold can create four inverted inserts at a time, so multiple copies of the mold were printed to expedite the insert creation process (FIG. 6). To form the wells of the inverted insert, another piece was 3D-printed using the same procedure above. The piece resembles a table, with four downward projections in the middle of the insert molds. Each projection allows the agarose to solidify around it, forming the well for the cell or DNA solution (FIG. 7). Finally, an insert pusher was created to help remove solidified inserts from the mold. It was designed to be slightly smaller than the insert size and can push the inserts out of the mold.

### Example 3—Making the Inverted Inserts

A range of UltraPure LMP (Low Melting Point) agarose concentrations were tested, 1%-2%, to make the inverted inserts. First, the tape was placed on the bottom or smooth side of the insert mold, and the mold was put on ice. Depending on the experiment, a 1% agarose solution was made in either DAF H₂O or 1×TE [10 mM Tris-HCl, 1 mM EDTA] pH 8. Water was used when the dye was being tested, but when DNA was used in the insert, TE was used. To make the inverted insert, 1% agarose solution was added to each section (FIG. 6A). The top of the mold (FIG. 6B) was placed on top of the insert mold to form the wells. Once the agarose in the mold solidified, the top was removed carefully so as not to damage the integrity of the wells. To test the inverted inserts, 120 μL of 1× loading dye with glycerol [6× loading dye (100 mL): 60 g glycerol, 0.06 M EDTA pH 8, bromophenol blue and xylene cyanole (Eastman Kodak Company, Rochester, NY)] solution was added to the wells. The glycerol increases the density of the solution and prevents the agarose that will be added to the top of the insert from sinking into the well and mixing with the solution. Finally, liquid agarose was added to the top of each insert to encase the well and finish the inverted insert. The structural integrity of the inverted insert and the ease of use were tested for each agarose concentration (Table 1). Three different concentrations of agarose solution were tested to determine which would be best for creating the inverted inserts. The initial criteria for testing were ease of use, fluorescence migration, solidification, and structural integrity.

TABLE 1
Determination of the best percentage of
agarose for making an inverted insert.

| Agarose | | | | |
|---|---|---|---|---|
| Conc. | | Fluorescence | Solidifies | Structural |
| (%) | Easy to Use | Migration | Easily | Integrity |
| 1.0 | Yes | Slight migration | Yes | Poor |
| 1.5 | Yes | No migration | Yes | Good |
| 2.0 | Hard to pipette | No migration | Yes, but | Great |
| | after initial | | almost too | |
| | melting due to | | quickly | |
| | the viscosity | | | |

### Example 4—Analysis of the DNA Within the Insert: Large DNA Molecules

Pulse Field Gel Electrophoresis (PFGE) [Bio-rad, Hercules, CA, USA] was used to determine whether the S. cerevisiae DNA remained full length and if the DNA remained in the inverted insert. 1% PFC (Pulsed Field Certified) agarose in 0.5×TBE buffer [45 mM Tris-borate, 1 mM EDTA] was solidified, and the samples were added to the wells. The gel was run using the auto algorithm (Molecular weight: 200-2200 kb, Temperature: 14° C., Gradient: 6.0 V/cm, Run time: 28 hr 59 min, Angle: 120°, initial switch time: 24.03 s, final switch time 3 min 48.48 s). The gel was stained with SYBR™ Gold Nucleic Acid Gel Stain (Thermo Fisher Scientific, Waltham, MA, USA) for 20 min, and images were taken using a blue light transilluminator and Canon EOS T7i camera (Melville, NY, USA).

### Example 5—Analysis of the DNA within the Insert: Small DNA Molecules

Lambda DNA (500 ng/μL) was digested with NEB Acc65I in 1×NEBuffer 3.1 (New England Biolabs, Ipswich, MA, USA) to determine if small DNA fragments remained in the inverted insert. The solution was incubated at 37° C. for two hours, and 4 μL of 0.5 M EDTA was added to each tube to stop the reaction. The DNA solution was added to the inverted insert with 1× loading dye, and agarose was added to the top. The inserts were stored for three weeks at 4° C. to give the DNA time to diffuse through the 2% agarose inverted insert if it was going to diffuse. After three weeks, a 1% agarose gel in 1×TAE [40 mM Tris, 20 mM Acetic Acid, 1 mM EDTA] buffer was run using the solution from the inverted inserts with loading dye. The gel was run at 100 V for 120 minutes before being stained and imaged with the same protocol.

### Example 6—Analysis of the DNA within the Insert: Low Melting Point Plug Inserts

Plug inserts were made using the methods listed in Schwartz et al. (1984, Cell, 37 (1): 67-75). A plug mold was designed in AutoCAD and printed using the Ultimaker 3D printer. Three gel concentrations were studied: 0.5%, 0.8%, and 1.0%. All low melting point (LMP) agarose samples were prepared with 1×TE and heated in conical tubes to near boiling temperature to ensure all the agarose dissolved. The prepared agarose was then cooled to 42° C., while a DNA solution consisting of 100 μL stock lambda DNA (500 ng/μL) and 200 μL of DAF 1×TE was brought up to the same temperature. Both solutions were mixed and transferred to a plug mold (7 mm width×3 mm length×7 mm height). The samples were cooled at room temperature until solidified; then, they were transferred to an Eppendorf tube and submerged in DAF 1×TE for over 2 weeks. The plug inserts were removed from the solution, placed into a 2 mL Eppendorf tube, and heated to 70° C. to melt the insert. Then, pre-warmed (42° C.) beta agarase (New England Biolabs, Ipswich, MA, USA) solution was added and incubated at 42° C. The DNA concentration was measured with an Implen Nanophotometer P330 (Munich, Germany). The solution that the insert was stored in was also measured to determine how much DNA diffused through the LMP plug insert into the solution. Additionally, a second experiment was run where lambda DNA was stored in the plug inserts for 2 weeks, digested with agarase, and then run on a 1% agarose gel for 2 hr at 100 V. A standard was run to quantify the amount of DNA in each lane.

### Example 7—Growing Saccharomyces cerevisiae

Next, a cell solution needed to be added to the inverted insert to determine if the S. cerevisiae DNA remains inside the inverted insert. First, a single colony of S. cerevisiae cells (ATCC 9080) from a plate was grown in 10 mL of YPD [10 g Bacto-yeast extract (BD Biosciences, Franklin Lakes, NJ, USA), 20 g Bacto-peptone (VWR, Radnor, PA, USA), 960 mL H₂O, 40 mL sterile 50% glucose] in a 100 mL flask. The cells were grown overnight at 37° C. with vigorous shaking. After overnight inoculation, 2 mL of the culture was added to 200 mL of YPD in a 2 L flask. This solution was then grown for 24 hours at 30° C. with vigorous shaking. Next, the culture was split into 50 mL centrifuge tubes and spun at 3000 rpm for 5 minutes in the accuSpin 1R centrifuge (Fisher Scientific). After the initial spin, the pellet was suspended in 0.05 M EDTA pH 7.5 in ⅕^{th} the original volume. Once added, the solution was spun at the same parameters as before, and the supernatant was poured off. The cells were washed with 0.05 M EDTA pH 7.5, and the solution was centrifuged again. The supernatant was poured off, and then 0.125 M EDTA pH 7.5 was added to resuspend the pellet into a cell solution. The cell solution was ready to be added to the inverted inserts.

### Example 8—Insert Creation and Cell Lysis in the Insert

Once the S. cerevisiae cell solution was ready, 2% inverted inserts were created using the same method in Example 3. Instead of using a loading dye solution, the S. cerevisiae cell solution was added to the wells. To maximize the amount of DNA in the inserts, 170 μL of the cell solution was added to each well and then covered with agarose. The inverted inserts were stored in a 50 mL conical tube. In each tube, a 7.5% beta-mercaptoethanol in 0.5 M EDTA pH 7.5 with Zymolase 20T (15.5 μl of 10 mg/ml Zymolase/mL of agarose solution) was added to cover all the inserts. These inserts were stored overnight at 4° C. The following day, NDSK (0.01 M Tris, 0.5 M EDTA, 1% N-lauroylsarcosine pH 9.5, and 2 mg/mL proteinase K) was added to complete the cell lysis. The inserts can then be stored at 4° C. until needed.

### Example 9—Developing a 3D-Printed Mold for Creating an Inverted Insert

Previous work (e.g., Schwartz et al., 1984, Cell, 37(1):67-75) created agarose plug inserts using an agarose insert mold with the agarose cell solution poured into the mold. The agarose solidified, and the plug inserts were removed using a glass rod upon solidification. Rather than store the DNA within the agarose, we flipped the insert and inverted it so the cell solution remained in the solution, and the agarose was on the outside, like a Gusher candy. To make these new inserts, a different mold needed to be created. The mold was designed in AutoCAD, exported into Cura to set the printing settings, and printed using an Ultimaker 3 printer. Several parts needed to be created (FIG. 6). The base of the mold (FIG. 6A) held the agarose solution. It was left open at the bottom and top. The tape was added to the bottom of the base, the mold was placed on ice, and the 1200 μl agarose solution was added. The top (FIG. 6B) part was placed on the mold to create the wells. Once the DNA/cell/loading dye solution was added to the wells, liquid agarose (tempered to 42° C.) was added to fully surround the liquid in agarose to make the inverted insert. A pusher (FIG. 6C) was designed to push out the inverted insert without breaking it. Then, a box (FIG. 6D) to hold the insert was designed so that when you cut off the top of the insert, it would be at the right height to pipet out the solution.

### Example 10—Development of the Inverted Inserts

The optimal agarose concentration for the inserts had to be determined to allow restriction enzymes and lysis solution to diffuse into the insert but prevent the large DNA molecules from migrating out of the inverted insert. Agarose concentrations from 1-2% were tested based on the following criteria (Table 1): ease of making the inverted inserts, fluorescence migration, solidification, and structural integrity. The inserts were tested with loading dye and YOYO-1-stained lambda DNA using the above-mentioned method. The 1% agarose inverted inserts were easy to work with and solidified quickly; however, they were flimsy and difficult to handle. To test the amount of DNA diffusing from the insert, YOYO-1-labeled lambda DNA was added to the center of the insert and imaged using a Blue light transilluminator and a Canon Rebel T7i camera. Over time, the fluorescent DNA diffused through the insert. For 2% agarose inverted inserts, they were structurally sound, solidified quickly, and there was no fluorescent migration; however, they were not easy to make. While the agarose solidified to make the wells in the device, the remaining agarose was kept warm. The 2% agarose solution became more viscous while waiting to make the wells in the insert (even while keeping the agarose warm). While this made it difficult to use the 2% agarose solution for this set of inverted inserts, this could be an agarose percentage to use in the future. The 1.5% agarose inverted inserts were quick to solidify and structurally sound; no fluorescent migration was noted, and the agarose solution was easy to work with. Therefore, the inverted inserts were created using a 1.5% agarose solution for the remainder of the experiments.

### Example 11—Dynamic Range of DNA Sizes in the Inverted Insert

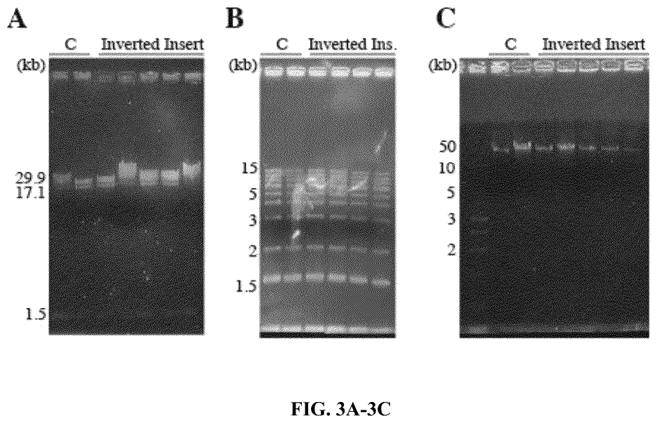

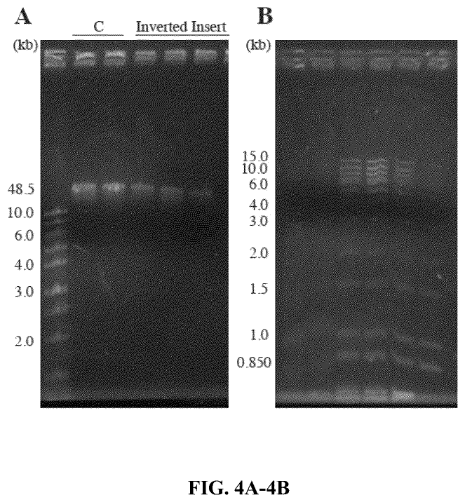

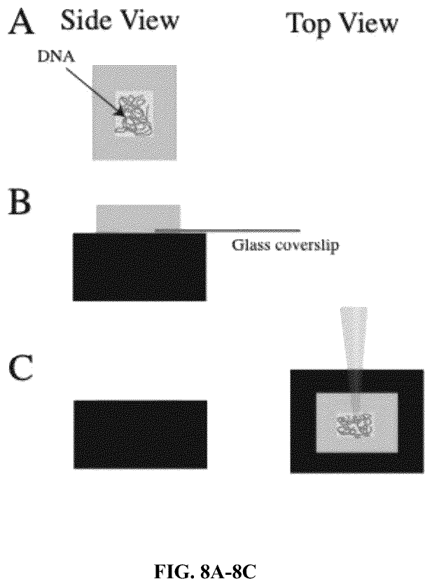

To test what DNA sizes remained in the inverted inserts over time, lambda DNA (48.5 kb), 1 kb plus ladder (1.5 to 15 kb in size), and lambda digested with Acc651 (1.5, 17.1, 29.9 kb fragment lengths) were added to the center well while making the inverted inserts. The inserts were stored in 1×TE buffer for 1 week. Then, the insert was placed in a holder, and the top was cut off with a glass coverslip (FIG. 8). The solution was removed using a pipette and stored in an Eppendorf tube. The samples were run on a 1% agarose gel in 0.5×TBE buffer for 2 hr at 100 V to determine if the DNA remained full length. The gels were stained with SYBR Gold for 30 min and then imaged with a Canon T7i Rebel camera and an Invitrogen blue light transilluminator. The DNA (lambda, lambda digested with Acc65I, and the 1 kb plus ladder (1.5 to 15 kb)) remained intact (FIG. 3). For the 1 kb ladder, the gels were run to separate fragments from 1.5-15 kb in size. The smaller sizes ran off the gel due to its running time. Future experiments can be done with smaller fragments to determine their retention in the inverted inserts and test different agarose concentrations to determine what DNA sizes remain. In order to test if the DNA remains full length for long storage times, some inverted inserts were stored for 1 year to determine if the DNA remained full length. In FIG. 4, the lambda and 1 kb plus ladder remained full length (≥1.5 kb) after 1 year of storage.

### Example 12—Comparison Between the Plug Insert and Inverted Insert

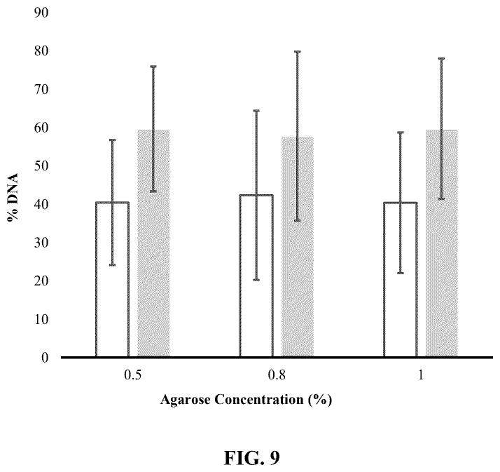

Plug inserts were developed previously to prevent the breakage of long DNA while the cells were stored in the agarose and lysed. The agarose protected the DNA from breakage during the lysis. One drawback of the plug insert is that the DNA is in agarose, not solution. The second drawback is that DNA near the edge of the insert will diffuse out of the insert and into the solution. Different low melting point agarose concentrations were tested to determine how much DNA remains in the insert over time. Three inserts for 0.5% agarose were tested with lambda DNA in each insert and immersed in DAF 1×TE for over 2 weeks. The insert was removed and placed into a 2 mL Eppendorf round bottom tube. The DNA concentration in the TE solution was measured using the Denovix DS-11 FX+. The insert was heated to 70° C. to melt the insert. Pre-warmed beta agarase (42° C.) was added to the melted insert, and the reaction was incubated at 42° C. for 2 hr. The amount of DNA in the insert was measured. FIG. 9 shows the average percentage of DNA in the solution (gray bars) or agarose (white bars). For all 3 agarose concentrations, 40% of DNA remained in the insert. In a second experiment, the plug inserts (after being stored in 1×TE for 2 weeks) were melted at 70° C., digested with agarase, run on a 1% agarose gel for 2 hr at 100 V, stained with SYBR gold, and quantified against a standard solution. The amount recovered was 40+10% of the original amount (N=10 inserts). The amount of DNA leaving the insert was likely due to DNA near the surface diffusing out of the insert and into the solution due to the high surface area to volume ratio. The surface area for the plug insert was 182 mm², and the area of the insert was 147 mm³. The insert size was based on the loading region for the elution and concentration device developed previously (21, 22).

In comparison, lambda DNA was added to the 1.5% inverted inserts and stored in the buffer for 2 weeks. The amount of lambda DNA was measured with a spectrophotometer (Denovix DS-11 FX+) and was 5500±900 ng or 64% of the original amount (8700 ng; N=8). The loss of DNA was most likely due to the agarose filling in the top of the inverted insert and slightly mixing with the DNA solution. The average volume recovered from the inverted insert was 137 μl.

### Example 13—Development of Saccharomyces cerevisiae Inverted Inserts

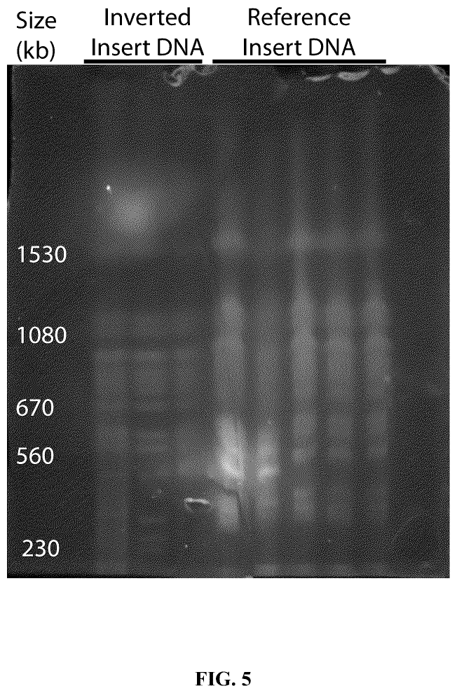

S. cerevisiae cells were used in the inverted inserts to show that cells could be lysed and the DNA could remain behind in the inverted inserts. Using the same method to make inverted inserts, the cells were added to the inverted insert, and then the agarose was added to the top. The cells were lysed in the inverted inserts and stored in EDTA for long-term storage at 4° C. for 3 weeks. The top of the insert was cut off using a glass slide, exposing the DNA solution in the well. The solution was removed with a wide bore pipette tip and added to a 1% pulsed field certified (PFC) agarose gel. As a control, S. cerevisiae was also added to plug inserts and ran on the gel as a control. The gel was run using the Auto Algorithm (200-2,200 kb; 6 V/cm, switch time: 24.03 s to 3 min 48.48 s; run time: 28 hr and 59 min; 0.5×TBE). The gel was stained with SYBR gold and imaged with a blue light transilluminator and Canon camera. As shown in FIG. 5, the DNA remains intact while stored in the inverted insert. The inverted insert protected DNA during cell lysis and kept the DNA molecules intact in solution while being stored for an extended period of time.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. An encapsulation device for protecting and storing DNA molecules during cell lysis, the encapsulation device comprising:
an agarose shell;
a central well defined within the agarose shell, the central well configured to receive a liquid sample comprising cells and/or the DNA molecules; and
a sealing layer of agarose disposed over the central well to encapsulate the liquid sample within the central well,
wherein the encapsulation device is configured such that, upon lysis of the cells within the liquid sample, the DNA molecules remain intact and in solution within the central well for subsequent recovery without melting the agarose shell.

2. The encapsulation device of claim 1, wherein the agarose shell has an agarose concentration between about 0.5% and about 3% (w/v).

3. The encapsulation device of claim 1, wherein the agarose shell has a thickness of about 0.2 centimeter (cm) to about 0.5 cm.

4. The encapsulation device of claim 1, wherein the agarose shell has a thickness sufficient to prevent diffusion of the DNA molecules into the agarose shell.

5. The encapsulation device of claim 1, wherein the agarose shell has a rectangular prism shape with a height ranging from about 1 cm to about 2 cm, a width ranging from about 0.5 cm to about 2 cm, and a length ranging from about 0.5 cm to about 2.5 cm.

6. The encapsulation device of claim 1, wherein the central well has a volume of about 140 microliters (μL) to about 200 μL.

7. The encapsulation device of claim 1, wherein the central well has a depth of about 0.5 cm to about 2 cm.

8. The encapsulation device of claim 1, wherein the sealing layer of agarose has a thickness of about 0.3 cm to about 1 cm.

9. The encapsulation device of claim 1, wherein the DNA molecules that remain intact and in solution have a length of about 100 kilobases (kb) to about 1.5 megabases (Mb).

10. A method of isolating DNA molecules, the method comprising:
providing the encapsulation device of claim 1 containing a liquid sample comprising cells and/or the DNA molecules in the central well;
contacting the encapsulation device with one or more lysis reagents to lyse the cells within the central well while the agarose shell remains intact; and
recovering the DNA molecules in liquid form from the central well without melting the agarose shell.

11. The method of claim 10, wherein recovering the DNA molecules comprises removing a portion of the sealing layer and removing the liquid sample from the central well.

12. The method of claim 10, wherein recovering the DNA molecules comprises poking a hole in the insert with a blunt end syringe.

13. The method of claim 10, further comprising rinsing the recovered DNA molecules with a buffer comprising one or both of Tris and EDTA.

14. The method of claim 10, further comprising storing the encapsulation device with the liquid sample in the central well at about 4° C. for at least one week prior to recovery.

15. A method of making an encapsulation device for protecting and storing DNA molecules during cell lysis, the method comprising:
providing a mold configured to form a shell, the mold defining one or more cavities;
applying a seal to a bottom opening of the one or more cavities;
introducing an agarose solution into the one or more cavities;
positioning a mold insert in the one or more cavities containing the agarose solution, the mold insert having a protrusion sized to define a central well;
cooling the agarose solution to solidify and form the shell;
removing the mold insert from the one or more cavities, thereby forming the central well;
dispensing a liquid sample comprising cells and/or DNA molecules into the central well; and
applying a sealing layer of agarose to encapsulate the liquid sample,
wherein the resulting encapsulation device is configured for subsequent recovery of the DNA molecules from the central well without melting the agarose shell.

16. The method of claim 15, wherein the mold is a rectangular mold.

17. The method of claim 15, wherein applying the sealing layer of agarose comprises applying the sealing layer at a temperature of about less than 45° C.

18. The method of claim 15, further comprising removing the seal from the bottom opening after cooling the agarose solution.

19. The method of claim 15, further comprising removing the solidified shell from the mold after removing the mold insert from the one or more cavities.

20. The method of claim 15, further comprising, after applying the sealing layer of agarose, storing the encapsulation device in a buffer at about 4° C.
